(19) United States
(12) Patent Application Publication
(10) Pub. No.: US 2021/0370069 A1
(43) Pub. Date: Dec. 2, 2021

(54) Implantable Stimulator with Embedded Receiver Relay

(71) Applicant: Micron Medical LLC, Boca Raton, FL (US)

(72) Inventors: Benjamin Speck, Boca Raton, FL (US); Graham Patrick Greene, Boca Raton, FL (US)

(21) Appl. No.: 17/403,089

(22) Filed: Aug. 16, 2021

Related U.S. Application Data
(63) Continuation of application No. 16/845,384, filed on Apr. 10, 2020, now abandoned.

(51) Int. Cl. A61N 1/36; A61N 1/378; A61N 1/372

(52) U.S. Cl. CPC A61N 1/36125; A61N 1/37223; A61N 1/3787

(57) ABSTRACT

An implantable stimulator includes a housing, a plurality of electrodes positioned along the housing, and a printed circuit board positioned along the housing. The printed circuit board includes control circuit components, a plurality of electrode connector pads, an antenna, and a receiver relay positioned along the printed circuit board such that a portion of the receiver relay overlaps with the antenna.

### RELATED APPLICATIONS

This application is a Continuation Application of U.S. application Ser. No. 16/845,384, filed on Apr. 10, 2020, which is hereby incorporated by reference in its entirety.

### TECHNICAL FIELD

This disclosure relates to an implantable stimulator, and more particularly, to an implantable stimulator with an embedded receiver relay to improve the RF energy transmission to electrodes of the stimulator without the addition of separate components.

### BACKGROUND

Neural modulation of tissue in the body by electrical stimulation has become an important type of therapy for chronic disabling conditions, such as pain, movement control, involuntary movement, dystonia, urinary and fecal incontinence, sexual dysfunction, vascular insufficiency, heart arrhythmia and a multitude of other neural based compromised modalities. Electrical stimulation of nerve bundles has been used commercially in the United States since the 1970s. Implanted electrodes are used to pass pulsatile electrical currents of controllable frequency, pulse width and amplitudes to surrounding tissue once implanted in the body. The electrical field may cross over certain neural elements, typically axons, and can selectively activate varying diameters of axons, with positive therapeutic benefits. A variety of therapeutic intra-body electrical stimulation techniques may be utilized to treat neuropathic conditions utilizing implanted electrodes connected to a source of power, such as a battery powered implanted pulse generator or wireless receiver, in the spinal column or surrounding areas, including the dorsal horn, dorsal root ganglia, dorsal roots, dorsal column fibers or peripheral nerve bundles leaving the dorsal column or brain, such as vagus-, occipital-, trigeminal, hypoglossal-, sacral-, ulnar-, median, radial-, cluneal, ilioguinal, tibial, and coccygeal nerves.

A transmitting antenna may be positioned on the body of the user, for example, as part of a wearable device wrapped about a portion of the user's body, to deliver radio frequency (“RF”) energy to a wireless receiver embedded in an implanted stimulation device in the body. Prior art implantable stimulation devices, or implantable stimulators, may include a receiving antenna element of various configurations that receives RF energy and subsequently transmits such energy to attached electrodes of the implantable stimulator, as illustrated in U.S. Pat. No. 10,179,244.

Prior art implanted stimulators may include a steering stylet placed into an inner lumen of the implantable stimulator body, which could, in some embodiments, be utilized to assist with guidance in routing the implantable stimulator through tissue to a distant target location in proximity of nerve tissue within the user's body. After the implantable stimulator has been implanted in the body, the steering stylet can be removed from the inner lumen, and a receiver relay may be inserted into the inner lumen. The receiver relay may be used to enhance transmission of RF energy from the transmitting antenna to a receiving antenna in the implantable stimulator. Once in place, the receiver relay may be sutured, glued, or crimped in place to ensure that it does not migrate outside of the inner lumen area of the implantable stimulator.

If the implantable stimulator placement needs to be adjusted, the receiver relay must be removed from the lumen, and then the steering stylet can then be re-inserted into the lumen and used to guide the implantable stimulator to the new target location. Once the implantable stimulator has been properly re-positioned, the stylet may be removed from the lumen, and the receiver relay can then be re-inserted into the lumen. This process may be repeated multiple times while searching for optimal placement of the electrodes.

### SUMMARY

In general, this disclosure relates to an implantable stimulator that includes a receiver relay printed directly onto a flexible circuit board substrate that is within the body of the implantable stimulator to improve RF energy transmission.

In accordance with one aspect, an implantable stimulator includes a housing, a plurality of electrodes positioned inside the housing, and a printed circuit board positioned inside the housing. The printed circuit board includes control circuit components, a plurality of electrode connector pads, an antenna, and a receiver relay positioned along the printed circuit board such that a portion of the receiver relay overlaps with the antenna to facilitate energy transfer.

In accordance with another aspect, an implantable stimulator includes a housing having a proximal end and a distal end. A plurality of electrodes is positioned inside the housing proximate the distal end of the housing. A printed circuit board is positioned inside the housing and has a plurality of layers including control circuit components placed on the outermost layers, an antenna having a proximal end and a distal end, with the distal end being proximate the plurality of electrodes, and a metallic receiver relay having a proximal end and a distal end. The receiver relay has a length longer than a length of the antenna, and does not have a direct physical electrical connection with the control circuit components and the antenna. The distal end of the receiver relay is proximate the distal end of the antenna, and the proximal end of the receiver relay extends toward the proximal end of the housing beyond the proximal end of the antenna.

In accordance with a further aspect, a kit for use in transmitting RF energy to an implantable stimulator includes a plurality of implantable stimulators, wherein each implantable stimulator includes a housing, a plurality of electrodes positioned inside the housing, and a printed circuit board that includes control circuit components, a receiving antenna, and a receiver relay positioned along the printed circuit board such that a portion of the receiver relay overlaps with the antenna. At least some of the implantable stimulators have a housing length different than a housing length of at least some other of the implantable stimulators. At least some of the implantable stimulators have an antenna length different than an antenna length of at least some other of the implantable stimulators. At least some of the implantable stimulators have a receiver relay length different than a receiver relay length of at least some other of the implantable stimulators.

In accordance with yet another aspect, a method of providing RF energy to the implantable tissue stimulator includes implanting the implantable stimulator in tissue of a body, wherein the implantable stimulator includes a housing, a plurality of electrodes positioned inside the housing, and a printed circuit board positioned in the housing, and the printed circuit board includes control circuit components, a receiving antenna, and a receiver relay positioned along the printed circuit board such that a portion of the receiver relay overlaps with the antenna; and positioning a transmitting antenna on a surface of the tissue of body such that the transmitting antenna is proximate the receiver relay.

Additional aspects, configurations, embodiments and examples are described in more detail below.

### DESCRIPTION OF DRAWINGS

Certain devices are described below with reference to the accompanying figures.

FIG. 1 is a schematic elevation view of an implantable stimulator with a receiving antenna and a receiver relay embedded within the internal flexible printed circuit board.

FIG. 2 is a schematic elevation view of layers of the printed circuit board of the implantable tissue stimulator of FIG. 1.

FIG. 3 is a schematic plan view of the printed circuit board of the implantable tissue stimulator of FIG. 1.

The figures referred to above are not drawn necessarily to scale and should be understood to provide representative examples, illustrative of the principles involved. Some features of the implantable stimulator with a receiver relay depicted in the drawings have been enlarged or distorted relative to others to facilitate explanation and understanding. The same reference numbers are used in the drawings for similar or identical components and features shown in various alternative embodiments. Implantable stimulators with embedded receiver relays as disclosed herein would have configurations and components determined, in part, by the intended application and environment in which they are used.

### DETAILED DESCRIPTION

Certain improvements to implantable stimulators are described herein. The term “approximately” as used herein is meant to mean close to, or about a particular value, within the constraints of sensible commercial engineering objectives, costs, manufacturing tolerances, and capabilities in the field of implantable stimulator manufacturing and use. Similarly, the term “substantially” as used herein is meant to mean mostly, or almost the same as, within the constraints of sensible commercial engineering objectives, costs, manufacturing tolerances, and capabilities in the field of implantable stimulator manufacturing and use.

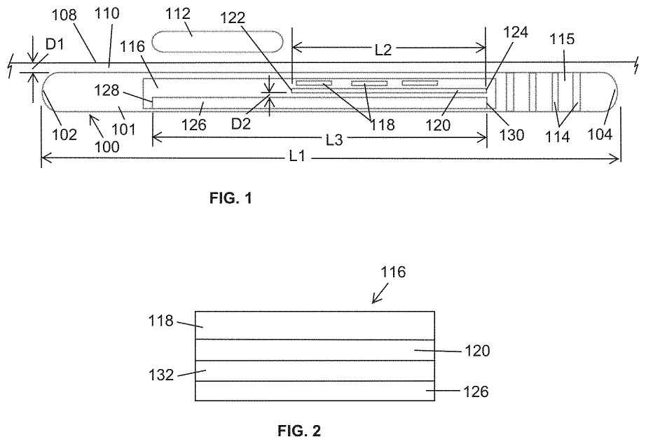

FIG. 1 shows an example of an implantable stimulator 100 having a housing 101, which is configured to be implanted within a patient's body for delivering electrical therapy to tissues within the body. Housing 101 may have a proximal end 102 and a distal end 104, and a length L1. In certain embodiments, L1 may be between approximately 10 cm and approximately 60 cm.

Housing 101 may have an exterior design that provides strength and a smooth profile for optimal insertion and performance within the tissue of the patient's body. For example, housing 101 may be laminated (e.g., catheter lamination) or molded (e.g., overmolded or insert molded) of a polymer material around various internal components of the implantable stimulator 100. Accordingly, implantable stimulator 100 may be referred to as a monolithic device with electronic components secured to one small, flat substrate, and which may be delivered to the targeted location within the body through the inner lumen of an introducer or needle.

The implantable tissue stimulator 100 may be implanted beneath a surface 108 of tissue 110 within the body at a distance D1. In certain embodiments, D1 may be between approximately 0.5 cm and approximately 10 cm.

A transmitting antenna 112 may be positioned proximate surface 108. In certain embodiments, transmitting antenna 112 may be part of a wearable device configured to be wrapped around a portion of a user's body. The transmitting antenna 112 may be configured to transmit RF energy through tissue 110 to the receiver within the implantable stimulator 100.

A plurality of electrodes 114 may be positioned inside housing 101 along a certain length of the implantable stimulator 100 and proximate to the distal end 104 of the implantable stimulator. Electrodes 114 may be spaced from one another by spacers 115. In the illustrated embodiment, the implantable stimulator 100 is shown with four electrodes 114. In other embodiments, the implantable stimulator 100 may include eight electrodes 114. It is to be appreciated that the implantable stimulator 100 is not restricted to embodiments with four or eight electrodes 114, and that suitable numbers of electrodes 114 for the implantable stimulator 100 will become readily apparent to those skilled in the art, given the benefit of this disclosure.

The implantable stimulator 100 may include a printed circuit board 116 in housing 101. Printed circuit board 116 may be a flexible laminate structure that can bend with the implantable stimulator 100 as it is inserted into tissue 110. In certain embodiments, printed circuit board 116 may be completely embedded within housing 101 of the implantable stimulator 100.

The printed circuit board 116 may include a plurality of components 118 that are configured to operate a control circuit of printed circuit board 116. The control circuit may be used to manage the electrical operations of the implantable stimulator 100. Exemplary components 118 include resistors, capacitors, and diodes. Printed circuit board 116 may be operably connected to electrodes 114 to ensure that the RF energy from transmitting antenna 112 can be transferred to electrodes 114.

The RF energy from the transmitting antenna 112 may be received by a receiving antenna 120 formed as a layer of printed circuit board 116. The receiving antenna 120 may include a proximal end 122 and a distal end 124. The distal end 124 of receiving antenna 120 may be positioned proximate electrodes 114. The receiving antenna 120 may have a length L2. In certain embodiments, L2 may be between approximately 4 cm and approximately 12 cm.

To enhance the transmission of RF energy from transmitting antenna 112 to the receiving antenna 120, a receiver relay 126 may be positioned as a layer of printed circuit board 116. The receiver relay 126 may be physically separate from electrical connections with other elements of printed circuit board 116 including components 118 and antenna 120. Receiver relay 126 may have a proximal end 128 and a distal end 130, and a length L3. In certain embodiments, L3 may be between approximately 8 cm and approximately 30 cm.

Providing receiver relay 126 as a separate layer helps ensure that receiver relay 126 is precisely positioned within printed circuit board 116. The manufacturing process used to create printed circuit board 116 helps ensures precise dimensions and placement of receiver relay 126 horizontally in the X and Y planes of printed circuit board 116. The stack-up of the laminated, or adhesively secured, layers of printed circuit board 116 helps ensure precise placement of the layer of relay receiver 126 vertically in the Z plane of printed circuit board 116, which helps ensure that relay receiver 126 is separated from receiving antenna 120 by a distance D2. In certain embodiments, D2 may be between approximately 0.0254 cm and approximately 0.1000 cm.

Receiver relay 126 may enhance reception of the RF energy transmitted by transmitting antenna 112, while minimizing specific absorption rate (“SAW”) and transmitter energy consumption. Receiver relay 126 may enhance reception of RF energy in cases where the implantable stimulator 100 is placed such that it is unfavorable for reception of RF energy from transmitting antenna 112. For example, receiver relay 126 many enhance reception in situations where the implantable stimulator 100 is implanted deep in tissue 110, i.e., when distance D1 is more than 2 cm. In other embodiments, receiver relay 126 many enhance reception when receiving antenna 120 is placed at an angle with respect to transmitting antenna 112, when receiving antenna 120 includes a bend or curvature along its length, or when the implantable stimulator 100 is placed in a location in tissue 110 that is unfavorable for placement of transmitting antenna 112, or any other scenario where the direct transmission of RF energy from transmitting antenna 112 to receiving antenna 120 is hampered.

In operation, the receiver relay 126 may pick up transmitted RF energy just under surface of the skin 108 where transmitting antenna 112 is placed, and propagate the RF energy along the length of receiver relay 126 from proximal end 128 to distal end 130, where the electric field may be polarized and optimally aligned, and where distal end 130 is in close proximity to receiving antenna 120. Thus, RF energy is coupled to receiving antenna 120 independent of its curvature, angle, or implanted depth within tissue 112.

The performance of the receiver relay 126 may be affected by the length L3 of receiver relay 126, which may be determined based on the distance that the RF energy needs to travel from just under surface 108 at the location of transmitting antenna 112 to receiving antenna 120.

Additionally, the electrical length of receiver relay 126 may enhance the RF resonances at the receiving antenna 120 and its ability to receive RF energy. It is to be appreciated that the electrical length of receiver relay 126 is equal to length L3 divided by the electromagnetic wavelength at the RF transmitting frequency, in the effective medium of tissue 110.

Further, the performance of receiver relay 126 may be affected by the longitudinal alignment of distal end 130 of receiver relay 126 and distal end 124 of receiving antenna 120.

Embedding the receiver relay 126 within printed circuit board 116 during its manufacture eliminates the need for a surgeon to insert a receiver relay into the implantable stimulator 100 after implantation in tissue 110 of the body, and also eliminates the need to remove the receiver relay in the event that the implantable stimulator 100 needs to be relocated, which can help prevent problems with the proper placement of receiver relay 126.

Additionally, forming receiver relay 126 as part of printed circuit board 116 may help ensure proper longitudinal alignment of receiver relay 126 and receiving antenna 120, which may help ensure the efficiency of coupling power between receiver relay 126 and receiving antenna 120.

Since receiver relay 126 is embedded in the implantable stimulator 100, it cannot be removed or exchanged after the implantable stimulator 100 is in position in tissue 110. Thus, it is to be appreciated that it would be advantageous for a surgeon to have a plurality of the implantable stimulators 100 ahead of time having housings 101 with different lengths L1, with receiving antennas 120 of different lengths L2, and receiver relays 126 of different lengths L3. Thus, the surgeon could select an implantable stimulator 100 having a housing with a desired length L1 or within a desired range of lengths L1, a receiving antenna 120 with a desired length L2 or within a desired range of lengths L2, and a receiver relay 126 with a desired length L3 or within a desired range of lengths L3. The surgeon's selection may be based upon the anatomy of the patient; the location of the target nerve is in tissue 110 (i.e., where electrodes 114 would lie), and where receiver relay 126 would need to lie within tissue 110. One advantage of planning and selecting the proper implantable stimulator 100 would be to ensure that neither electrodes 114 nor receiver relay 126 are positioned over a body part that experiences bending or flexing.

Accordingly, in some embodiments, a kit including a plurality of implantable stimulators 100 may be provided. In some embodiments, the plurality of implantable stimulators 100 in the kit may each have different housing lengths L1. In such an embodiment, the lengths L1 may be in a first range from approximately 10 cm to approximately 60 cm. In other embodiments, sets of implantable stimulators 100 may be provided, with each set having a particular housing length L1 that is different from each of the other sets. It is to be appreciated that any number of sets of implantable stimulators 100A-N could be provided.

Thus, for example, three sets of implantable stimulators 100A, 100B, and 100C could be provided with set 100A having implantable stimulators 100 with a first housing length L1A, set 100B having implantable stimulators 100 with a second housing length L1B that is different from first housing length L1A, and third set 100C having implantable stimulators 100 with a third housing length L1C that is different from first housing length L1A and second housing length LIB.

The implantable stimulators 100 of the kit may also be provided with antennas 120 having different antenna lengths L2. In some embodiments, implantable stimulators 100 may be provided with antennas 120 having antenna lengths L2 in a second range from approximately 4 cm to approximately 12 cm. In other embodiments, each of sets 100A-N may be provided with antennas 120 having a particular antenna length L2 that is different from each of the other sets. Thus, for example, set 100A could have a first antenna length L2A, set 100 B could have a second antenna length L2B that is different from first antenna length L2A, and set 100C could have a third antenna length L2C that is different from first antenna length L2A and from second antenna length L2B.

In certain embodiments, each set 100A-N could have antennas 120 with different antenna lengths L2, or subsets of antenna lengths L2. Thus, for example, set 100A could have antennas with three sets of antenna lengths L2A, L2B, and L2C; set 100B could also have antennas with three sets of antenna lengths L2A, L2B, and L2C; and set 100C could also have antennas with three sets of antenna lengths L2A, L2B, and L2C.

The implantable stimulators 100 of the kit may also be provided with receiver relays 126 having different receiver relay lengths L3. In some embodiments, implantable stimulators 100 may be provided with receiver relays 126 having receiver relay lengths L3 in a third range from approximately 8 cm to approximately 30 cm. In other embodiments, each of sets 100A, 100 B, and 100C may be provided with receiver relays 126 having a particular receiver relay length L3 that is different from each of the other sets. Thus, set 100A could have a first receiver relay length L3A, set 100 B could have a second receiver relay length L3B that is different from first receiver relay length L3A, and set 100C could have a receiver relay length L3C that is different from first receiver relay length L3A and from second receiver relay length L3B.

In certain embodiments, each set 100A-N could have a receiver relay with different receiver relay lengths L3, or subsets of receiver relay lengths L3. Thus, for example, set 100A could have receiver relays 126 with three sets of receiver relay lengths L3A, L3B, and L3C; set 100B could also have antennas with three sets of receiver relay lengths L3A, L3B, and L3C; and set 100C could also have antennas with three sets of receiver relay lengths L3A, L3B, and L3C.

It is to be appreciated that the kit could be provided with any number of implantable stimulators 100 or sets of implantable stimulators 100A-N; with any number of housing lengths L1, or range of housing lengths L1-N, or subsets of housing lengths L1-N; any number of antenna lengths L2, or range of antenna lengths L2-N, or subsets of antenna lengths L2-N; and any number of receiver relay lengths L3, or range of receiver relay lengths L3-N, or subsets of receiver relay lengths L3-N.

It is to be appreciated that when forming a kit of implantable stimulators, there is no limit on the number of implantable stimulators that can be included in each set and/or subset of implantable stimulators, and that there is no limit on the number of sets and/or subsets of implantable stimulators, housing lengths L1, antenna lengths L2, or receiver relay lengths L3.

In certain embodiments receiver relay 126 and receiving antenna 120 may be oriented within printed circuit board 116 such that proximal end 128 of receiver relay 126 is proximate proximal end 102 of housing 101, distal end 130 of receiver relay 126 extends beyond proximal end 122 of receiving antenna 120, and a portion of receiver relay 126 overlaps with at least a portion of receiving antenna 120. In certain embodiments, distal end 130 of receiver relay 126 may be positioned at the same point along printed circuit board 116 as distal end 124 of receiving antenna 120.

A schematic illustration of exemplary layers of printed circuit board 116 is shown in FIG. 2. As noted above, a layer of printed circuit board 116 may be formed as receiver relay 126, which may be a metallic layer. In certain embodiments, receiver relay 126 may be formed of copper or silver. An insulator 132 may be formed as a layer above receiver relay 126. Insulator 132 may be formed of polyimide (PI), polyester (PET), or polyether ether ketone (PEEK). Receiving antenna 120 may be formed as a layer of printed circuit board 116 above insulator 132. Receiving antenna 120 may be formed of copper or silver. One or more components 118 (e.g., a capacitor) may be positioned above receiving antenna 120, as seen in greater detail below.

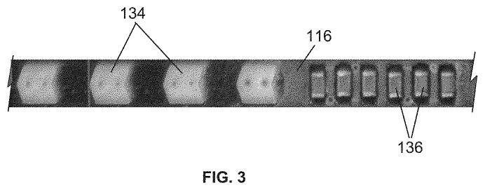

As illustrated in FIG. 3, printed circuit board 116 may include a plurality of electrode connector pads 134, each of which may be connected to a corresponding electrode 114 by way of wires, pins, solder, conductive epoxy, or other suitable connection means (not shown). In the illustrated embodiment, four electrode connector pads 134 are seen on printed circuit board 116, which may be connected to the four electrodes 114 shown in FIG. 1. It is to be appreciated that any number of electrode connector pads 134 can be provided on printed circuit board 116, with the number of electrode connector pads 134 corresponding to the number of electrodes 114. As noted above, a plurality of components such as capacitors 118 may be positioned on printed circuit board 116.

Several alternative embodiments and examples have been described and illustrated herein. A person of ordinary skill in the art would appreciate the features of the individual embodiments, and the possible combinations and variations of the components. A person of ordinary skill in the art would further appreciate that any of the embodiments could be provided in any combination with the other embodiments disclosed herein. It is understood that the invention may be embodied in other specific forms without departing from the spirit or central characteristics thereof. The present examples and embodiments, therefore, are to be considered in all respects as illustrative and not restrictive, and the invention is not to be limited to the details given herein. Terms “top,” “upper,” “bottom,” “lower,” “left,” “right,” and the like, as used herein, are intended for illustrative purposes only and do not limit the embodiments in any way. When used in description of a method or process, the term “providing” (or variations thereof) as used herein means generally making an article available for further actions, and does not imply that the entity “providing” the article manufactured, assembled, or otherwise produced the article. Nothing in this specification should be construed as requiring a specific three dimensional orientation of structures in order to fall within the scope of this invention, unless explicitly specified by the claims. Additionally, the term “plurality,” as used herein, indicates any number greater than one, either disjunctively or conjunctively, as necessary, up to an infinite number. Accordingly, while the specific embodiments have been illustrated and described, numerous modifications come to mind without significantly departing from the spirit of the invention and the scope of protection is only limited by the scope of the accompanying claims.

What is claimed is:

1. An implantable stimulator comprising:
a housing;
a plurality of electrodes positioned inside the housing; and
a printed circuit board positioned inside the housing and including:
control circuit components;
a plurality of electrode connector pads;
an antenna; and
a receiver relay positioned along the printed circuit board such that a portion of the receiver relay overlaps with the antenna.

2. The implantable stimulator of claim 1, wherein the receiver relay is a layer of the printed circuit board.

3. The implantable stimulator of claim 1, wherein the receiver relay is a metallic layer of the printed circuit board.

4. The implantable stimulator of claim 1, wherein the receiver relay is free of electrical connections with the control circuit components and the antenna.

5. The implantable stimulator of claim 1, wherein:
the electrodes are positioned proximate a distal end of the housing;
the antenna has a proximal end and a distal end, the distal end of the antenna being proximate the electrodes; and
the receiver relay has a proximal end that is proximate a proximal end of the housing, and a distal end that extends beyond the proximal end of the antenna.

6. The implantable stimulator of claim 1, wherein a length of the receiver relay is longer than a length of the antenna.

7. The implantable stimulator of claim 1, wherein four electrodes are positioned inside the housing.

8. The implantable stimulator of claim 1, wherein eight electrodes are positioned inside the housing.

9. An implantable stimulator comprising:
a housing having a proximal end and a distal end;
a plurality of electrodes positioned inside the housing proximate the distal end of the housing; and
a printed circuit board positioned inside the housing and having a plurality of layers including:
control circuit components;
an antenna having a proximal end and a distal end, the distal end being proximate the plurality of electrodes; and
a receiver relay having a proximal end and a distal end, a length longer than a length of the antenna, and being physically separate from electrical connections with the control circuit components and the antenna, the distal end of the receiver relay being proximate the distal end of the antenna, and the proximal end of the receiver relay extending toward the proximal end of the housing beyond the proximal end of the antenna.

10. The implantable stimulator of claim 9, wherein four electrodes are positioned inside the housing.

11. The implantable stimulator of claim 9, wherein eight electrodes are positioned inside the housing.

12. A kit for use in transmitting RF energy to an implanted stimulator comprising:
a plurality of implantable stimulators, wherein:
each implantable stimulator includes a housing, a plurality of electrodes positioned inside the housing, and a printed circuit board that includes control circuit components, a receiving antenna, and a receiver relay positioned along the printed circuit board such that a portion of the receiver relay overlaps with the antenna,
at least some of the implantable stimulators have a housing length different than a housing length of at least some other of the implantable stimulators,
at least some of the implantable stimulators have an antenna length different than an antenna length of at least some other of the implantable stimulators, and
at least some of the implantable stimulators have a receiver relay length different than a receiver relay length of at least some other of the implantable stimulators.

13. The kit of claim 12, wherein the plurality of stimulators includes a plurality of sets of stimulators, each set including implantable stimulators having a housing length that is different than a housing length of each of the other sets, each set including stimulators having an antenna length that is different than an antenna length of each of the other sets, and each set having a receiver relay length that is different than a receiver relay length of each of the other sets.

14. A method of providing RF energy to an implantable stimulator comprising:
implanting a stimulator in tissue of a body, wherein the implantable stimulator includes a housing, a plurality of electrodes positioned inside the housing, and a printed circuit board positioned in the housing and including control circuit components, a receiving antenna, and a receiver relay positioned along the printed circuit board such that a portion of the receiver relay overlaps with the antenna; and
positioning a transmitting antenna on a surface of the tissue of the body such that the transmitting antenna is proximate the receiver relay.

15. The method of claim 14, wherein:
the electrodes are positioned proximate a distal end of the housing;
the antenna has a proximal end and a distal end, the distal end of the antenna being proximate the electrodes; and
the receiver relay has a proximal end that is proximate a proximal end of the housing, and a distal end that extends beyond the proximal end of the antenna.

16. The method of claim 14, wherein the receiver relay is a metallic layer of the printed circuit board.

17. The method of claim 14, wherein the receiver relay is free of electrical connections with the control circuit components and the antenna.

18. The method of claim 14, wherein a length of the receiver relay is longer than a length of the antenna.

19. The method of claim 14, wherein four electrodes are positioned inside the housing.

20. The method of claim 14, wherein eight electrodes are positioned inside the housing.
